(19) United States
(12) Patent Application Publication
(10) Pub. No.: US 2023/0210934 A1
(43) Pub. Date: Jul. 6, 2023

(54) LYCIUM BARBARUM LEAF POLYSACCHARIDE RICH IN GALACTURONIC ACID AND PREPARATION METHOD AND USE THEREOF

(71) Applicant: Zhejiang University, Hangzhou (CN)

(72) Inventors: Shiguo Chen, Hangzhou (CN); Chengxiao Yu, Hangzhou (CN); Xinxin Hu, Hangzhou (CN); Jianle Chen, Hangzhou (CN); Haibo Pan, Hangzhou (CN); Xingqian Ye, Hangzhou (CN); Donghong Liu, Hangzhou (CN); Tian Ding, Hangzhou (CN)

(73) Assignee: Zhejiang University, Hangzhou (CN)

(21) Appl. No.: 17/652,210

(22) Filed: Feb. 23, 2022

(30) Foreign Application Priority Data
Jan. 5, 2022 (CN) 202210003768.9

(51) Int. Cl. A61K 36/815; C08B 37/00

(52) U.S. Cl. CPC A61K 36/815; C08B 37/0003

(57) ABSTRACT

Provided are a lycium barbarum leaf polysaccharide rich in galacturonic acid and a preparation method and use thereof. The method includes: mixing a lycium barbarum leaf and an acetone aqueous solution to obtain a mixture, and performing a fading treatment on the mixture to obtain a faded lycium barbarum leaf; extracting the faded lycium barbarum leaf with a chelating agent solution to obtain an extract solution; subjecting the extract solution to an alcohol precipitation with ethanol to obtain an alcohol precipitate; and subjecting the alcohol precipitate to an alcohol washing, a water redissolution, a dialysis and a drying in sequence to obtain the lycium barbarum leaf polysaccharide rich in galacturonic acid.

### CROSS REFERENCE TO RELATED APPLICATION

This application claims priority to Chinese Application No. 202210003768.9, filed on Jan. 5, 2022, the content of which is incorporated herein by reference in its entirety.

### TECHNICAL FIELD

The present disclosure relates to the technical field of biomedicines, in particular to a lycium barbarum leaf polysaccharide rich in galacturonic acid and a preparation method and use thereof.

### BACKGROUND ART

Lycium barbarum leaf, also known as lycium barbarum bud tea, has effects such as nourishing kidney, benefiting essence, clearing away heat, improving eyesight, and delaying aging. It has a history of more than two thousand years of medicinal use in China. The lycium barbarum leaf is rich in polysaccharides, polyphenols, betaine, vitamins and minerals. However, compared with lycium barbarum, the research on the chemical composition of lycium barbarum leaf has not been paid much attention. Studies have shown that polysaccharide is one of the most important components in lycium barbarum leaf. The polysaccharide in lycium barbarum leaf has a structure similar to that in lycium barbarum, and has a content as high as 10.23%, which is even higher than that in lycium barbarum. The lycium barbarum leaf polysaccharide, i.e, polysaccharide in lycium barbarum leaf, is mainly composed of neutral sugars, further contains galacturonic acid and protein, is a main active substance in lycium barbarum leaf, and has antioxidant effects.

The current methods for extraction of lycium barbarum leaf polysaccharide mostly focus on high-temperature water extraction, which makes it impossible to damage cell wall of lycium barbarum leaf enough to fully release the lycium barbarum leaf polysaccharide in the cell wall, resulting in low galacturonic acid content.

### SUMMARY

An object of the present disclosure is to provide a lycium barbarum leaf polysaccharide rich in galacturonic acid and a preparation method and use thereof. The lycium barbarum leaf polysaccharide prepared by the method according to the present disclosure is rich in galacturonic acid and has the ability to regulate human intestinal flora.

In order to achieve the above object, the present disclosure provides the following technical solutions.

Disclosed is a method for preparing a lycium barbarum leaf polysaccharide rich in galacturonic acid, comprising:

mixing a lycium barbarum leaf and an acetone aqueous solution to obtain a mixture, and performing a fading treatment on the mixture to obtain a faded lycium barbarum leaf;

extracting the faded lycium barbarum leaf with a chelating agent solution to obtain an extract solution;

subjecting the extract solution to an alcohol precipitation with ethanol to obtain an alcohol precipitate; and

subjecting the alcohol precipitate to an alcohol washing, a water redissolution, a dialysis and a drying in sequence to obtain the lycium barbarum leaf polysaccharide rich in galacturonic acid.

In some embodiments, the acetone aqueous solution has a volume fraction of not less than 60%, and a ratio of the lycium barbarum leaf to the acetone aqueous solution is in the range of 1 g:(5-10) mL.

In some embodiments, the fading treatment is performed at a temperature of 25-35° C. for 3-5 times, each time for 1-2 h.

In some embodiments, the chelating agent solution comprises a chelating agent selected from the group consisting of 1,2-cyclohexanediamine tetraacetic acid and ethylenediaminetetraacetic acid.

In some embodiments, the chelating agent in the chelating agent solution has a concentration of 0.04-0.06 mol/L, and a ratio of the faded lycium barbarum leaf to the chelating agent solution is in the range of 1 g:(20-40) mL.

In some embodiments, the chelating agent solution is prepared by a process comprising the following steps:

mixing the chelating agent with a sodium acetate buffer to obtain a mixed solution; and

regulating the mixed solution to a pH of 6.5-7.0 to obtain the chelating agent solution.

In some embodiments, the extracting is performed at a temperature of 20-30° C. for 3-5 h.

In some embodiments, a volume ratio of the ethanol to the extract solution is in the range of (2-4):1.

The present disclosure also provides a lycium barbarum leaf polysaccharide rich in galacturonic acid prepared by the method as described in the above technical solutions, wherein the galacturonic acid has a content of 50-70 mol %.

The present disclosure also provides use of the lycium barbarum leaf polysaccharide rich in galacturonic acid as described above in the preparation of formulations for regulating human intestinal flora.

The present disclosure provides a method for preparing a lycium barbarum leaf polysaccharide rich in galacturonic acid, comprising the following steps: mixing a lycium barbarum leaf and an acetone aqueous solution to obtain a mixture, and performing a fading treatment on the mixture to obtain a faded lycium barbarum leaf; extracting the faded lycium barbarum leaf with a chelating agent solution to obtain an extract solution; subjecting the extract solution to an alcohol precipitation with ethanol to obtain an alcohol precipitate; and subjecting the alcohol precipitate to an alcohol washing, a water redissolution, a dialysis and a drying in sequence to obtain the lycium barbarum leaf polysaccharide rich in galacturonic acid. In the present disclosure, the chelating agent is used as an extractant, and could chelate with high-valent cations (mainly calcium ions) in the cell wall of the lycium barbarum leaf through a chelating effect, which could break the connection between calcium bridge and the cell wall and accelerate the dissolution of pectinic acid connected with the calcium bridge, thereby obtaining a pectin-like polysaccharide with a high galacturonic acid content. The lycium barbarum leaf polysaccharide prepared by the method according to the present disclosure is rich in galacturonic acid and has an effect of regulating human intestinal flora. In addition, the method according to the present disclosure is simple to operate and could obtain a lycium barbarum leaf polysaccharide with a high galacturonic acid content without further separation and purification.

### DETAILED DESCRIPTION OF THE EMBODIMENTS

The present disclosure provides a method for preparing a lycium barbarum leaf polysaccharide rich in galacturonic acid, comprising the following steps:

mixing a lycium barbarum leaf and an acetone aqueous solution to obtain a mixture, and performing a fading treatment on the mixture to obtain a faded lycium barbarum leaf;

extracting the faded lycium barbarum leaf with a chelating agent solution to obtain an extract solution;

subjecting the extract solution to an alcohol precipitation with ethanol to obtain an alcohol precipitate; and

subjecting the alcohol precipitate to an alcohol washing, a water redissolution, a dialysis and a drying in sequence to obtain the lycium barbarum leaf polysaccharide rich in galacturonic acid.

In the present disclosure, a lycium barbarum leaf is mixed with an acetone aqueous solution, and the resulting mixture is subjected to a fading treatment to obtain a faded lycium barbarum leaf In some embodiments, the lycium barbarum leaf includes at least one selected from the group consisting of Ningxia lycium barbarum leaf, Chinese lycium barbarum leaf, Xinjiang lycium barbarum leaf and black fruit lycium barbarum leaf In some embodiments, the lycium barbarum leaf is dried, crushed and sieved in sequence to obtain a lycium barbarum leaf powder, and the obtained lycium barbarum leaf powder is mixed with the acetone aqueous solution, and the resulting mixture is then subjected to a fading treatment to obtain a faded lycium barbarum leaf. In some embodiments, the drying is performed by sun. There is no particular limitation to the crushing, and any crushing may be used as long as the obtained lycium barbarum leaf powder could meet the particle size requirements. In some embodiments, the sieving is performed by a sieve with 30-50 meshes, and a lycium barbarum leaf powder passing through the sieve is collected for use. In some embodiments, the acetone aqueous solution has a volume fraction of not less than 60%, preferably 60-80%. In some embodiments, the ratio of the lycium barbarum leaf to the acetone aqueous solution is in the range of 1 g:(5-10) mL, preferably 1 g:(5-8) mL, and more preferably 1 g:(5-6) mL.

In some embodiments, the lycium barbarum leaf is immersed in the acetone aqueous solution for fading treatment. In some embodiments, the fading treatment is performed at a temperature of 25-35° C., specifically at ambient temperature. In some embodiments, the ambient temperature refers to 25° C. In some embodiments, the fading treatment is performed for 3-5 times. After each fading treatment, the resulting reaction material is subjected to a solid-liquid separation, and a solid material collected therefrom is again immersed in the acetone aqueous solution for the fading treatment. There is no particular limitation to the method of solid-liquid separation, and any method of solid-liquid separation well known to those skilled in the art may be used. In some embodiments, the solid-liquid separation is performed by filtration using a filter cloth with 300-400 meshes. In some embodiments, each fading treatment is performed for 1-2 hours. In some embodiments, the fading treatment is carried out under stirring. There is no particular limitation to stirring rate, and any stirring rate well known to those skilled in the art may be used. In some embodiments, the fading treatment is carried out under the above-mentioned conditions, so that most of the pigment in the lycium barbarum leaf could be removed.

In the present disclosure, the obtained faded lycium barbarum leaf is extracted by a chelating agent solution to obtain an extract solution. In some embodiments, the chelating agent in the chelating agent solution includes at least one selected from the group consisting of 1,2-cyclohexanediamine tetraacetic acid (CDTA) and ethylenediaminetetraacetic acid (EDTA). In some embodiments, the chelating agent is a food-grade chelating agent. In some embodiments, the chelating agent in the chelating agent solution has a concentration of 0.04-0.06 mol/L, preferably 0.05 mol/L. In some embodiments, the chelating agent solution is prepared by a process comprising the following steps: mixing the chelating agent with a sodium acetate buffer to obtain a mixed solution and regulating the mixed solution to a pH of 6.5-7.0 to obtain the chelating agent solution. In some embodiments, the sodium acetate buffer has a concentration of 0.05-0.15 mol/L, preferably 0.1 mol/L. In some embodiments, the pH value is adjusted by an agent which is selected from the group consisting of hydrochloric acid and sodium hydroxide according to actual needs. In some embodiments, the chelating agent solution has a pH of 6.5-7.0, which could prevent a structure of pectin in the extract solution from being damaged due to a low pH. In some embodiments, the ratio of the faded lycium barbarum leaf to the chelating agent solution is in the range of 1 g:(20-40) mL, preferably 1 g:(25-30) mL.

In some embodiments, the faded lycium barbarum leaf is extracted by immersing in a chelating agent solution. In some embodiments, the extracting is performed at a temperature of 20-30° C., preferably 25-30° C. In some embodiments, the extracting is performed for 3-5 hours, preferably 3-4 hours. In some embodiments, the extracting is performed under stirring. There is no particular limitation to stirring rate, and any stirring rate well known to those skilled in the art may be used. In the present disclosure, during the extracting process, the chelating agent could continuously chelate with high-valent cations (mainly calcium ions) in the cell wall of the lycium barbarum leaf, and thus could break the connection between calcium bridge and the cell wall, thereby continuously releasing pectin-like polysaccharide connected with calcium ions and obtaining pectin-like polysaccharide with a high galacturonic acid content.

After the extracting, the resulting reaction material is subjected to a solid-liquid separation, and the liquid material collected therefrom is the extract solution. There is no particular limitation to the method of solid-liquid separation, and any method of solid-liquid separation well known to those skilled in the art may be used. In some embodiments, the solid-liquid separation is performed by filtration using a filter cloth with 300-400 meshes.

Subsequently, the obtained extract solution is subjected to an alcohol precipitation with ethanol to obtain an alcohol precipitate. In some embodiments, the volume ratio of the ethanol to the extract solution is in the range of (2-4):1, preferably (2-3):1. In some embodiments, the ethanol is anhydrous ethanol. In some embodiments, the extract solution is mixed with ethanol, and then kept stand for alcohol precipitation. In some embodiments, the alcohol precipitation is performed at a temperature of 25-35° C., and specifically at ambient temperature. In some embodiments, the alcohol precipitation is performed for 2-5 hours, preferably 3-4 hours.

In some embodiments, the resulting reaction material from the alcohol precipitation is subjected to a solid-liquid separation, and the solid material collected therefrom is the alcohol precipitate. There is no particular limitation to the mode of solid-liquid separation, and any mode of solid-liquid separation well known to those skilled in the art may be used. In some embodiments, the solid-liquid separation is performed by filtration using a filter cloth with 300-400 meshes.

In the present disclosure, the obtained alcohol precipitate is subjected to an alcohol washing, a water redissolution, a dialysis and a drying in sequence to obtain the lycium barbarum leaf polysaccharide rich in galacturonic acid. In some embodiments, the alcohol washing is performed by anhydrous ethanol. In some embodiments, the water redissolution is performed in distilled water, with an amount to ensure that the alcohol precipitate washed with an alcohol is completely redissolved to obtain a redissolved solution. In some embodiments, the dialysis is performed by a dialysis bag which has a molecular weight cutoff of 8000-14000 Da, preferably 10000 Da. In some embodiments, the dialysis is performed in a sodium chloride solution and distilled water in sequence. In some embodiments, the sodium chloride solution has a concentration of 0.05-0.2 mol/L, preferably 0.1-0.15 mol/L. Specifically, the redissolved solution obtained by water redissolution is subjected to a first dialysis with a sodium chloride solution as a dialysate, then subjected to a second dialysis with distilled water as a dialysate. In some embodiments, the first dialysis and the second dialysis are performed for 12-24 hours independently, preferably 20-24 hours. In some embodiments, the sodium chloride solution is used as the dialysate for dialysis to remove the chelating agent in the redissolved solution obtained by water redissolution, and distilled water is used as the dialysate for dialysis to remove sodium ions and chlorine ions introduced during the first dialysis. In some embodiments, the drying is freeze drying.

The present disclosure provides a lycium barbarum leaf polysaccharide rich in galacturonic acid prepared by the method as described in the above technical solutions. The lycium barbarum leaf polysaccharide provided by the present disclosure has a high content of galacturonic acid, which is 50-70 mol %, preferably 60-70 mol %. In some embodiments, the lycium barbarum leaf polysaccharide further includes fucose, rhamnose, arabinose, galactose and glucose. In some embodiments, a content of the fucose is 0.1-1 mol %. In some embodiments, a content of the rhamnose is 5-10 mol %. In some embodiments, a content of the arabinose is 10-15 mol %. In some embodiments, a content of the galactose is 5-10 mol %. In some embodiments, a content of the glucose is 5-10 mol %. In some embodiments, the lycium barbarum leaf polysaccharide of the present disclosure does not contain xylose and glucuronic acid.

The present disclosure provides use of the lycium barbarum leaf polysaccharide rich in galacturonic acid described in the above technical solutions in the preparation of formulations for regulating human intestinal flora. The lycium barbarum leaf polysaccharide of the present disclosure has the effect of regulating human intestinal flora, stimulating the growth of probiotics, especially the effect of promoting the growth of bifidobacteria and lactobacilli simultaneously.

The technical solutions of the present disclosure will be clearly and completely described below in conjunction with the examples of the present disclosure. Obviously, the described examples are only a part of the examples of the present disclosure, rather than all the examples. All other examples obtained by those skilled in the art based on the examples described herein without creative work shall fall within the scope of the present disclosure.

### EXAMPLE 1

(1) A lycium barbarum leaf (specifically Ningxia lycium barbarum leaf) dried by sun was crushed with a high-speed pulverizer, and sieved by a sieve with 50 meshes, to obtain a lycium barbarum leaf powder which passed through the sieve. The obtained lycium barbarum leaf powder was immersed in an acetone aqueous solution with a volume fraction of 80% at a ratio of the lycium barbarum leaf to the acetone aqueous solution of 1 g:5 mL, subjected to a fading treatment at ambient temperature (25° C.) under stiffing for 2 hours, and then filtered with a filter cloth with 300 meshes to obtain a filter cake. The filter cake was subjected to a fading treatment with the acetone aqueous solution for 3 times, to obtain a faded lycium barbarum leaf powder.

(2) A sodium acetate buffer with a concentration of 0.1 mol/L was prepared by using water as a solvent. The sodium acetate buffer was mixed with 1,2-cyclohexanediamine tetraacetic acid (CDTA) to obtain a mixture in which the CDTA had a concentration of 0.05 mol/L, and then the mixture was adjusted to have a pH value of 6.80 by using 1 mol/L hydrochloric acid to obtain a CDTA solution. The faded lycium barbarum leaf powder was immersed in the CDTA solution with a ratio of the faded lycium barbarum leaf powder to the CDTA solution of 1 g:30 mL, extracted at a temperature of 30° C. under stirring for 4 hours, and then filtered with a filter cloth with 300 meshes to obtain a filtrate, i.e. an extract solution.

(3) The extract solution was mixed with anhydrous ethanol with a volume ratio of the extract solution to the anhydrous ethanol of 1:3, then kept stand at ambient temperature for 4 hours, and then filtered with a filter cloth with 300 meshes to obtain a filter cake, i.e. an alcohol precipitate.

(4) The alcohol precipitate was washed by using anhydrous ethanol to obtain a solid material which was alcohol insoluble. The solid material was completely redissolved by distilled water to obtain a redissolved solution, and the redissolved solution was then dialyzed by a dialysis bag with a molecular weight cutoff of 10,000 Da. Specifically, the redissolved solution was firstly subjected to a dialysis in a sodium chloride solution with a concentration of 0.1 mol/L for 24 hours, and then subjected to a dialysis in distilled water for 24 hours to obtain a polysaccharide solution. The polysaccharide solution was freeze-dried to obtain a lycium barbarum leaf polysaccharide.

### COMPARATIVE EXAMPLE 1

A Lycium barbarum leaf polysaccharide was prepared according to the method of Example 1, except that the CDTA solution was replaced with a sodium carbonate solution. The sodium carbonate solution was prepared by the method as follows: a sodium borohydride buffer with a concentration of 25 mmol/L was prepared using water as a solvent, and mixed with sodium carbonate to obtain a sodium carbonate solution in which the sodium carbonate had a concentration of 0.05 mol/L.

### COMPARATIVE EXAMPLE 2

A Lycium barbarum leaf polysaccharide was prepared according to the method of Example 1, except that the CDTA solution was replaced with a sodium hydroxide solution. The sodium hydroxide solution was prepared by the method as follows: a sodium borohydride buffer with a concentration of 25 mmol/L was prepared using water as a solvent, and mixed with sodium hydroxide to obtain a sodium hydroxide solution in which the sodium hydroxide had a concentration of 0.1 mol/L.

### TEST EXAMPLE 1

The composition of monosaccharides in the lycium barbarum leaf polysaccharide prepared in Example 1 and Comparative Examples 1-2 was determined by ion chromatography, as follows:

1 mL of the lycium barbarum leaf polysaccharide aqueous solution with a concentration of 2 mg/mL was added into an ampoule, and then 1 mL of a trifluoroacetic acid aqueous solution with a concentration of 4 mol/L was added into the ampoule. The ampoule was sealed by using an alcohol blowtorch, and then hydrolysis was performed at a temperature of 110° C. for 8 hours. Then trifluoroacetic acid in the ampoule was removed by a nitrogen blowing, and the remaining material was dissolved in 6 mL of distilled water, filtered with a 0.22 μm micropore water-based membrane to obtain a sample to be tested, and then analyzed by an ion exchange chromatograph to quantify the monosaccharide composition by using a standard curve. The standard curve was prepared as follows:

10 μmol/L, 20 μmol/L, 40 μmol/L, 60 μmol/L and 80 μmol/L of standard solutions containing 9 kinds of monosaccharides (fucose, rhamnose, arabinose, galactose, glucose, mannose, xylose, galacturonic acid, glucuronic acid) were prepared respectively, filtered with a 0.22 μm micropore water-based membrane, and then injected into the ion exchange chromatograph together with the sample to be tested. Standard curves of the 9 kinds of monosaccharides were made with the concentration of monosaccharide as abscissa and peak area as ordinate, as follows:

Fucose: y=123.27x+4.76, R²=0.998; Rhamnose: y=115.72x+0.46, R²=0.999; Arabinose: y=167.20−1.921, R²=0.999; Galactose: y=227.49x+3.11, R²=0.997; Glucose: y=247.96x−1.63, R²=0.999; Mannose: y=161.10x−3.50, R²=0.993; Xylose: y=242.61x−6.73, R²=0.999; Galacturonic acid: y=95.17x−2.31, R²=0.997; and Glucuronic acid: y=86.89x−3.01, R²=0.993.

Table 1 shows the monosaccharide composition (in mol %) of the lycium barbarum leaf polysaccharide prepared in Example 1 and Comparative Examples 1-2. It can be seen from Table 1 that the content of galacturonic acid in lycium barbarum leaf polysaccharide extracted by the chelating agent in the inventive example is significantly higher than those extracted by sodium carbonate and sodium hydroxide.

Table 1 Monosaccharide composition (in mol %) of the lycium barbarum leaf polysaccharide prepared in Example 1 and Comparative Examples 1-2

TABLE 1

| | | Comparative | Comparative |
|---|---|---|---|
| | | Example 1 | Example 2 |
| | Example 1 | Polysaccharide | Polysaccharide |
| | Polysaccharide | extracted by | extracted by |
| Monosaccharide | extracted by the | sodium | sodium |
| species | chelating agent | carbonate | hydroxide |
| Fucose | 0.36 ± 0.10 | 0.591 ± 0.009 | 1.748 ± 1.173 |
| Rhamnose | 7.27 ± 0.04 | 24.133 ± 0.875 | 9.679 ± 1.109 |
| Arabinose | 10.58 ± 2.45 | 26.980 ± 0.280 | 20.447 ± 0.998 |
| Galactose | 8.72 ± 1.19 | 24.066 ± 0.248 | 16.158 ± 0.800 |
| Glucose | 8.15 ± 1.61 | 14.576 ± 0.326 | 46.103 ± 5.680 |
| Xylose | Ns | Ns | Ns |
| Galacturonic acid | 64.92 ± 0.49 | 8.410 ± 0.562 | 4.339 ± 1.660 |
| Glucuronic acid | Ns | 1.245 ± 0.357 | 1.526 ± 0.483 |

Note: “Ns” represents not detected.

### TEST EXAMPLE 2

A carbon-free medium was prepared, which consisted of 10 g/L of casein peptone, 2.5 g/L of yeast extract, 0.09 g/L of magnesium sulfate heptahydrate, 0.09 g/L of calcium chloride, 0.45 g/L of potassium dihydrogen phosphate, 0.45 g/L of dipotassium hydrogen phosphate, 0.9 g/L of sodium chloride, 1.5 g/L of sodium bicarbonate, 1.0 g/L of cysteine hydrochloride, 0.8 mg/L of azuresin, 10 mg/L of chlorohemine, 5.0 mg/L of Vitamin B2, 10.0 mg/L of Vitamin B6, 2.0 mg/L of Vitamin B7, 0.1 mg/L of Vitamin B12, 2.0 mg/L of folic acid and 5.0 mg/L of para-aminobenzoic acid. The carbon-free medium had a pH value of 7.20. 10 g of human feces and 90 mL of PBS buffer (pH 7.0) were mixed and homogenized to obtain a human feces homogenate with a concentration of 10% (w/v).

The 12yceum barbarum leaf polysaccharide prepared in Example 1 was dissolved in the carbon-free medium, and then the human feces homogenate was added thereto with a volume of 10% of the carbon-free medium. The resulting mixture was subjected to a fermenting reaction at a temperature of 37° C. under an anaerobic condition for 24 hours to obtain a fermented solution. The fermented solution was centrifuged to collect a feces sediment which was measured by using qPCR (Quantitative Real-time PCR) method to determine the content of Bifidobacterium, Lactobacillus plantarum, Escherichia coli and Bacteroides in the feces sediment. The specific steps were as follows:

Bifidobacterium, Lactobacillus plantarum, Escherichia coli and Bacteroides were cultivated by a liquid medium. The number of viable bacteria (CFU/mL) of each type of bacteria in the liquid medium after cultivation was determined by a plate coating method. The feces sediment and standard strains were subjected to a DNA extraction by a DNA extraction kit. Specifically, 0.3 μL of a 10 μmol/L corresponding primer aqueous solution of each bacteria was added into 5 μL of qPCR premix (SYBR Green I) (The primer was selected according to the literatures of: Gil-Sanchez, I., Cueva, C., Sanz-Buenhombre, M., Guadarrama, A., Moreno-Arribas, M. V., & Bartolome, B. (2018). Dynamic gastrointestinal digestion of grape pomace extracts: Bioaccessible phenolic metabolites and impact on human gut microbiota. Journal of Food Composition and Analysis, 68, 41-52), and then 3.4 μL of enzyme-free water and 1 μL of DNA template were added thereto. The resulting mixture was measured, and a standard curve was made with Ct value as abscissa and logarithm of the copy number as ordinate. The content of specific bacteria in the sample was quantified with the standard curve.

The standard curves of each bacteria and total bacteria were as follows:

Bifidobacterium: y=−0.295x+10.593, R²=0.998; Lactobacillus plantarum: y=−0.276x+10.718, R²=0.998; Escherichia coli: y=−0.250x+9.479, R²=0.987; Bacteroides: y=−0.287x+10.622, R²=0.989; and total bacteria: y=−0.214x+10.407, R²=0.998.

The above test group was record as a polysaccharide group. A blank control group experiment and an inulin group experiment were set up at the same time. Wherein, the blank control group experiment was the same as the polysaccharide group except that 13yceum barbarum leaf polysaccharide was not added in the blank control group experiment; and the inulin group experiment was the same as the polysaccharide group except that the 13yceum barbarum leaf polysaccharide was replaced with the same amount of inulin (from Dahlia, purchased from Aladdin company).

Table 2 shows the effects of the 13yceum barbarum leaf polysaccharide prepared in Example 1 on the contents of the four bacteria and total bacteria. It can be seen from Table 2 that compared with the blank control group, the 13yceum barbarum leaf polysaccharide extracted by the chelating agent in the inventive example could significantly increase the contents of two common probiotics, namely Bifidobacterium and Lactobacillus plantarum, and exhibit an intestinal prebiotic activity.

Table 2 The effects of the 13yceum barbarum leaf polysaccharide prepared in Example 1 on the contents of the four bacteria and total bacteria (in login (copy number/mL))

TABLE 2

| Bacterial | Blank control | Inulin | Polysaccharide |
|---|---|---|---|
| species | group | group | group |
| Bifidobacterium | 4.92 ± 0.14 | 6.31 ± 0.15 | 5.51 ± 0.10 |
| Lactobacillus | 3.85 ± 0.10 | 5.07 ± 0.21 | 4.66 ± 0.20 |
| plantarum | | | |
| Bacteroides | 7.26 ± 0.22 | 7.41 ± 0.06 | 7.72 ± 0.23 |
| Escherichia | 6.26 ± 0.58 | 6.21 ± 0.23 | 6.50 ± 0.07 |
| coli | | | |
| Total bacteria | 8.35 ± 0.30 | 8.82 ± 0.26 | 8.68 ± 0.19 |

The above are only the preferred embodiments of the present disclosure. It should be pointed out that for those skilled in the art, some improvements and modifications without departing from the principle of the present disclosure could be made, and these improvements and modifications shall also fall within the protection scope of the present disclosure.

1. A method for preparing a lycium barbarum leaf polysaccharide rich in galacturonic acid comprising:
mixing a lycium barbarum leaf and an acetone aqueous solution to obtain a mixture; performing a fading treatment on the mixture to obtain a faded lycium barbarum leaf;
extracting the faded lycium barbarum leaf with a chelating agent solution to obtain an extract solution;
subjecting the extract solution to an alcohol precipitation with ethanol to obtain an alcohol precipitate; and
subjecting the alcohol precipitate to an alcohol washing, a water redissolution, a dialysis and a drying in sequence to obtain the lycium barbarum leaf polysaccharide rich in galacturonic acid.

2. The method of claim 1, wherein the acetone aqueous solution has a volume fraction of not less than 60%, and a ratio of the lycium barbarum leaf to the acetone aqueous solution is in the range of 1 g:(5-10) mL.

3. The method of claim 1, wherein the fading treatment is performed at a temperature of 25-35° C. for 3-5 times, each time for 1-2 h.

4. The method of claim 1, wherein the chelating agent solution comprises a chelating agent selected from the group consisting of 1,2-cyclohexanediamine tetraacetic acid and ethylenediamine tetraacetic acid.

5. The method of claim 1, wherein the chelating agent in the chelating agent solution has a concentration of 0.04-0.06 mol/L, and a ratio of the faded lycium barbarum leaf to the chelating agent solution is in the range of 1 g:(20-40) mL.

6. The method of claim 1, wherein the chelating agent solution is prepared by a process comprising the following steps:
mixing the chelating agent with a sodium acetate buffer to obtain a mixed solution; and
regulating the mixed solution to a pH of 6.5-7.0 to obtain the chelating agent solution.

7. The method of claim 1, wherein the extracting is performed at a temperature of 20-30° C. for 3-5 h.

8. The method of claim 1, wherein a volume ratio of the ethanol to the extract solution is in the range of (2-4):1.

9. A lycium barbarum leaf polysaccharide rich in galacturonic acid prepared by the method of claim 1, wherein the galacturonic acid has a content of 50-70 mol %.

10. A method for regulating human intestinal flora, comprising administrating the lycium barbarum leaf polysaccharide rich in galacturonic acid of claim 9 to a subject in need thereof.

11. The method of claim 2, wherein the fading treatment is conducted at a temperature of 25-35° C. for 3-5 times, wherein a single fading treatment is conducted for 1-2 h.

12. The method of claim 4, wherein the chelating agent in the chelating agent solution has a concentration of 0.04-0.06 mol/L, and a ratio of the faded lycium barbarum leaf to the chelating agent solution is in the range of 1 g:(20-40) mL.

13. The method of claim 4, wherein the chelating agent solution is prepared by the following steps:
mixing the chelating agent with a sodium acetate buffer to obtain a mixed solution; and
regulating the mixed solution to a pH of 6.5-7.0 to obtain the chelating agent solution.

14. The lycium barbarum leaf polysaccharide rich in galacturonic acid of claim 9, wherein the acetone aqueous solution has a volume fraction of not less than 60%, and a ratio of the lycium barbarum leaf to the acetone aqueous solution is in the range of 1 g:(5-10) mL.

15. The lycium barbarum leaf polysaccharide rich in galacturonic acid of claim 9, wherein the fading treatment is performed at a temperature of 25-35° C. for 3-5 times, each time for 1-2 h.

16. The lycium barbarum leaf polysaccharide rich in galacturonic acid of claim 9, wherein the chelating agent solution comprises a chelating agent selected from the group consisting of 1,2-cyclohexanediamine tetraacetic acid and ethylenediamine tetraacetic acid.

17. The lycium barbarum leaf polysaccharide rich in galacturonic acid of claim 9, wherein the chelating agent in the chelating agent solution has a concentration of 0.04-0.06 mol/L, and a ratio of the faded lycium barbarum leaf to the chelating agent solution is in the range of 1 g:(20-40) mL.

18. The lycium barbarum leaf polysaccharide rich in galacturonic acid of claim 9, wherein the chelating agent solution is prepared by a process comprising the following steps:
mixing the chelating agent with a sodium acetate buffer to obtain a mixed solution; and
regulating the mixed solution to a pH of 6.5-7.0 to obtain the chelating agent solution.

19. The lycium barbarum leaf polysaccharide rich in galacturonic acid of claim 9, wherein the extracting is performed at a temperature of 20-30° C. for 3-5 h.

20. The lycium barbarum leaf polysaccharide rich in galacturonic acid of claim 9, wherein a volume ratio of the ethanol to the extract solution is in the range of (2-4):1.
